(19) United States
(12) Patent Application Publication
(10) Pub. No.: US 2009/0318555 A1
(43) Pub. Date: Dec. 24, 2009

(54) Treatment of Vertigo with Acetyl-L-Leucine

(75) Inventors: Pierre Fabre, Castres (FR); Christophe Przybylski, Villefranche de Lauragais (FR); Anne-Sophie Saurel, Toulouse (FR)

Correspondence Address: BIRCH STEWART KOLASCH & BIRCH, PO BOX 747, FALLS CHURCH, VA, 22040-0747, US

(73) Assignee: PIERRE FABRE MEDICAMENT, Boulogne-Billancourt (FR)

(21) Appl. No.: 12/310,908

(22) PCT Filed: Sep. 13, 2007
(86) PCT No.: PCT/IB2007/003644

§ 371 Date: Mar. 12, 2009

(30) Foreign Application Priority Data
Sep. 13, 2006 (FR) 06/07992

(51) Int. Cl. A61K 31/197

(52) U.S. Cl. 514/561

(57) ABSTRACT

The use of acetyl-L-leucine and the pharmaceutically acceptable salts of same for the manufacture of a medicament for the treatment of vertigo and other balance disorders. Advantageously, the acetyl-L-leucine is a 100% mixture.

The present invention relates to the use of acetyl-L-leucine and pharmaceutically acceptable salts of same for the manufacture of a medicament for the treatment of vertigo and other balance disorders.

The concept of neuroplasticity refers to a set of neurobiological mechanisms underlying CNS adaptations and reorganizations in response to environmental changes or as a consequence of attacks on CNS functional integrity. CNS plasticity is highly active during ontogenetic development and continues to be expressed in fully-mature adults.

Thus, in a wide variety of species, unilateral lesion of labyrinth afferents leads to a static syndrome, observed at rest, and a dynamic syndrome, which appears during the initiation or execution of movements of the head and body. Static syndrome encompasses oculomotor deficits (spontaneous vestibular nystagmus) and postural deficits (head tilt to the lesioned side, limb muscle tone asymmetry). A lesioned animal cannot stay upright and falls repeatedly on the lesioned side. This syndrome is the consequence of extreme disequilibrium of spontaneous activity of ipsilateral and contralateral vestibular nucleus (VN) neurons. Dynamic syndrome is expressed by severe deterioration of the vestibulo-ocular reflex, an effect responsible for poor eye stabilization during head movements as well as oscillopsia in man. These vestibulo-ocular deficits are associated with extreme changes in the ability to maintain equilibrium, reflecting significant deterioration of the vestibulo-spinal reflexes involved in head and limb control. Such behavioral data are also interpreted in terms of changes in the dynamic response properties of VN neurons located near the lesion.

Compensation for vestibular deficits reflects total or subtotal regression of the symptoms described above. Lacour (Contribution to the study of restoration of posturo-kinetic functions after labyrinthectomy in the monkey and the cat [Contribution à l'étude de la restauration des fonctions posturo-cinétiques après labyrinthectomie chez le singe et le chat], Ph.D. thesis [in French], Marseille (1981), 154 pp.) distinguished three characteristic stages in the monkey and the cat:
a critical phase with maximum disorders (first week post-lesion),
an acute phase of rapid but incomplete regression of the initial asymmetries,
a compensation phase (three weeks to several months) which leads to restoration of postural-locomotor and oculomotor functions.

Regression of all deficits indicates reequilibration of static and dynamic vestibulo-spinal and vestibulo-ocular influences and may arise from the more or less complete restoration of spontaneous activity of vestibular neurons near the lesion. Such VN reequilibration activity has been demonstrated electrophysiologically and confirmed by measurements of cellular energy metabolism using the labeled deoxyglucose technique.

The nature of the mechanisms by which spontaneous activity of deafferented vestibular neurons returns to near-normal levels is still unknown. However, it appears highly probable that neurochemical reorganization plays an important functional role (Darlington and Smith: Molecular mechanisms of recovery from vestibular damage in mammals: recent advances, Prog Neurobiol (2000), 62, 313-325; Darlington C L, Dutia M B, Smith P F: The contribution of the intrinsic excitability of vestibular nucleus neurons to recovery from vestibular damage, Eur J. Neurosci. (2002), 15, 1719-1727). Indeed, some studies have demonstrated the existence of post-lesion changes in VN neurotransmitter systems and changes in the time course of vestibular compensation have been noted after treatment with the agonists or antagonists of these transmitters and/or their receptors.

Study of the influence of drugs or pharmacological substances acting on vestibular deficit compensation regression and/or quality is of major interest in clinical medicine due to the relatively high frequency of vestibular pathologies, vertigo and disorders of posture and balance.

Acetyl-leucine in racemate form, marketed by Pierre Fabre Medicament as an anti-vertigo medicament under the name Tanganil®, is currently used successfully in the treatment of acute peripheral vertigo in clinical practice. Previous work by the inventors has shown that this substance considerably accelerates the regression of postural and kinetic deficit compensation in the cat, compared to untreated lesioned animals. The behavioral effects demonstrated include a significant (50%) shortening of the vestibular compensation time constant observed both after intravenous treatment (IV: 28 mg/kg) during the first three days post-lesion and after intra-osseous treatment (IO: 28 mg/kg) during the first 30 days postoperative (Lacour M, Pascalis O: Acetyl-DL-leucine and vestibular compensation: behavioral study [Acetyl-Dl-Leucine et compensation vestibulaire: étude comportementale], Le Cerebellum: Satellite symposium on the treatment of vertigo [in French], Paris (1992) and Pascalis O: Behavioral and electrophysiological approaches for vestibular deficit compensation in the cat: pharmacological mechanisms and treatment [Approches comportementale et électrophysiologique de la compensation des déficits vestibulaires chez le chat: mécanismes et traitements pharmacologiques], DEA Neurosciences [in French], Université de Provence, Marseilles (1990) 42 pp.).

Nevertheless, the development of molecules with antivertiginous properties and substances likely to act on the cellular/molecular mechanisms involved in functional restoration after a pathological attack on the vestibular system remains of significant interest in the fields of health and medicaments.

Within the scope of the present application, in order to demonstrate the particularly advantageous properties of the L isomer, the inventors used an established experimental model of animals having undergone unilateral vestibular neurectomy. The selected experimental model and protocol are recognized in the field of neurosensory research as targeting the study of disorders associated with vertigo crises.

Thus, the inventors were able to demonstrate the substantial effect of the acetyl-L-leucine enantiomer. Indeed, it arises from these results that the acetyl-L-leucine enantiomer provides all postural, locomotor and oculomotor functional restoration activity. For this reason, the acetyl-L-leucine enantiomer is a well-founded, particularly desirable and advantageous choice for the treatment of vertigo and related disorders.

Demonstration of the properties of acetyl-L-leucine is genuinely surprising at both quantitative and qualitative levels. Indeed, the inventors noted with the present experimental model that administration of the acetyl-D-leucine isomer does not provide any improvement compared to a placebo, whereas it appears that restorative activity is only provided by the acetyl-L-leucine isomer. The extent of the difference in activity between the two isomers is remarkable and all the more surprising since the racemate has been known and marketed for many years without anyone suspecting any difference in activity between the two constitutive isomers of the racemic mixture.

Consequently, the present invention relates to the use of acetyl-L-leucine and the pharmaceutically acceptable salts of same for the manufacture of a medicament for the treatment of vertigo and other balance disorders.

In a preferred embodiment of the invention, a mixture is used that comprises 95%-100% acetyl-L-leucine, advantageously a mixture with 96%-100% acetyl-L-leucine or a mixture with 97%-100% acetyl-L-leucine or a mixture with 98%-100% acetyl-L-leucine or a mixture with 99%-100% acetyl-L-leucine, even more advantageously a mixture with 100% acetyl-L-leucine.

Within the meaning of the present invention, “vertigo and other balance disorders” means, in particular, benign paroxysmal positional vertigo (BPPV); vestibular neuritis; vertigo related to Meniere's disease, Wallenberg's syndrome, cerebellar ischemia, perilymph fistula or acoustic neurinoma; or recurring vertigo of traumatic or toxic origin.

The present invention also relates to the use of acetyl-L-leucine and the pharmaceutically acceptable salts of same for the manufacture of a medicament for the restoration of postural, locomotor and oculomotor functions deteriorated by a vestibular lesion.

Within the scope of the present invention, acetyl-L-leucine or the pharmaceutically acceptable salts of same can be provided in any dosage form suited to oral, rectal, subcutaneous, topical, intravenous or intramuscular administration. All such dosage forms are prepared by techniques known by those persons skilled in the art at a suitable dosage in combination with typical pharmaceutically acceptable excipients. Advantageous administration forms are all forms suited to intravenous administration and all forms suited to oral administration, notably tablets, pills, granules, powders, hard capsules, soft capsules, gelatin capsules, lyophilized tablets, syrups, emulsions, suspensions, solutions and films.

When acetyl-L-leucine or the pharmaceutically acceptable salts of same are administered by intravenous route, the dose is advantageously 100 mg to 2 g per day without interruption.

When acetyl-L-leucine or the pharmaceutically acceptable salts of same are administered by oral route, the doses may be between 100 mg and 20 g or more per day, advantageously between 100 mg and 4 g per day.

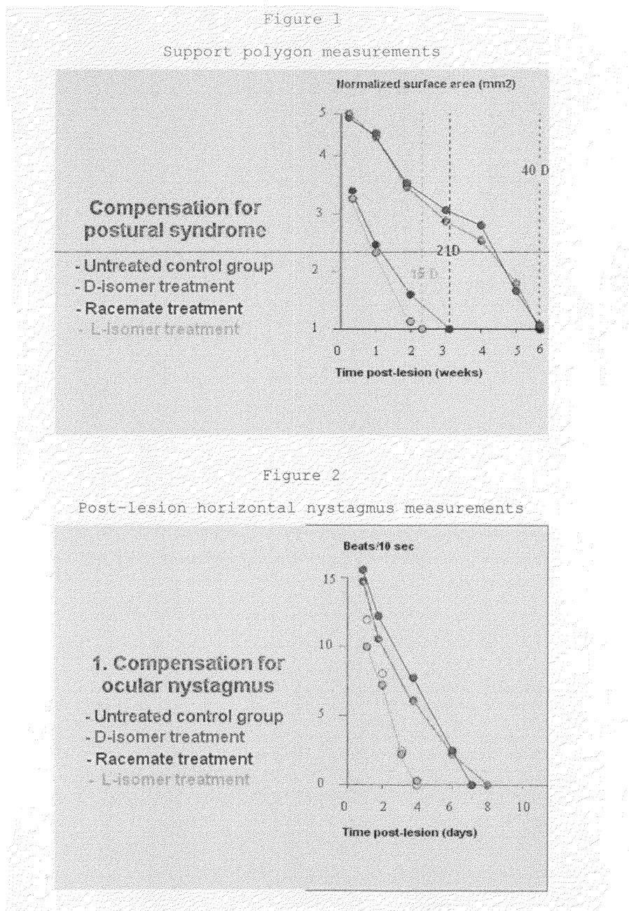

The examples and FIGS. 1 to 4 which follow illustrate the invention.

FIG. 1 represents compensation for postural syndrome in control animals (black plot), animals treated with acetyl-D-leucine (red plot), treated with acetyl-DL-leucine (green plot) and treated with acetyl-L-leucine (yellow plot) under the conditions described in example 1.

FIG. 2 represents compensation for ocular nystagmus in control animals (black plot), animals treated with acetyl-D-leucine (red plot), treated with acetyl-DL-leucine (green plot) and treated with acetyl-L-leucine (yellow plot) under the conditions described in example 1.

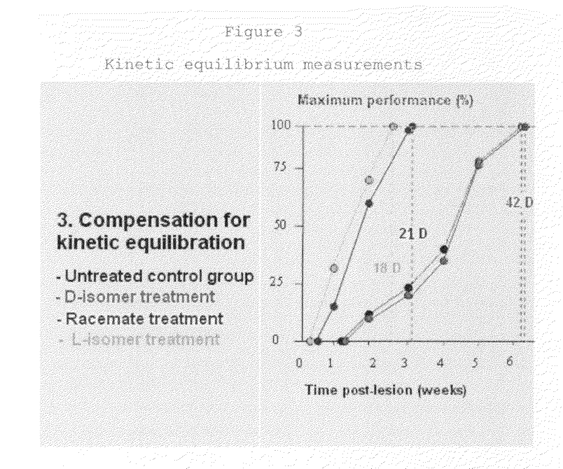

FIG. 3 represents compensation for kinetic equilibrium in control animals (black plot), animals treated with acetyl-D-leucine (red plot), treated with acetyl-DL-leucine (green plot) and treated with acetyl-L-leucine (yellow plot) under the conditions described in example 1.

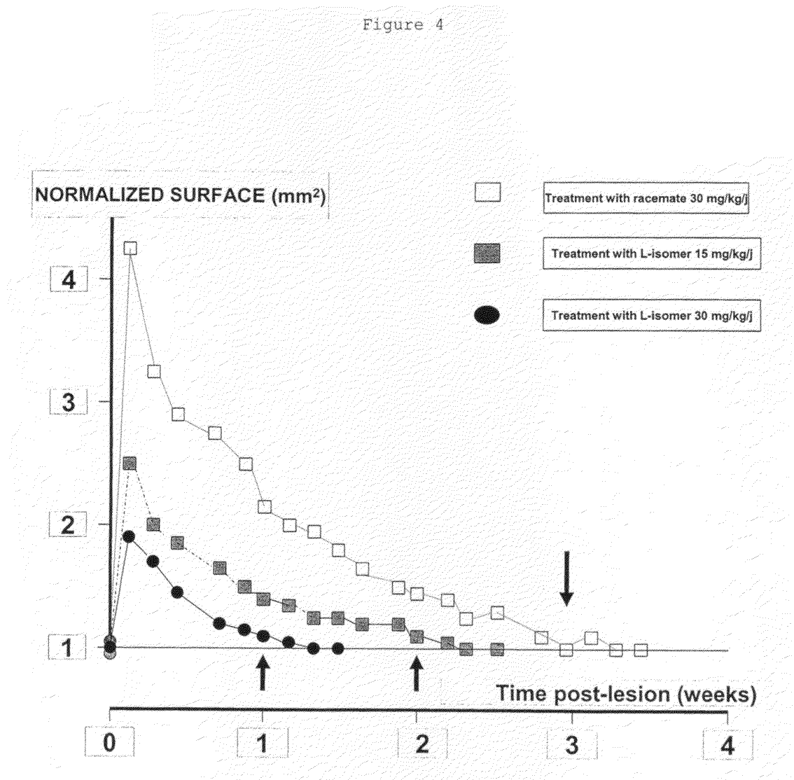

FIG. 4 represents compensation for postural syndrome in animals treated with acetyl-DL-leucine at 30 mg/kg per day (white squares) (white squares), with acetyl-L-leucine at 15 mg/kg per day (grey squares) and with acetyl-L-leucine at 30 mg/kg per day (black rounds) in the conditions described in example 2.

### EXAMPLE 1

### Effect of Acetyl-L-Leucine in a Unilateral Vestibular Neurectomy Model in the Cat

### 1.1. Protocol

### 1.1.1. Vestibular Neurectomy

The experiment involves 17 cats from the breeder IFA-CREDO (France).

The cats undergo a unilateral vestibular neurectomy on the left side.

Surgery is performed using a surgical microscope, under rigorously aseptic conditions, according to a translabyrinthine approach. After incision of the tissues located behind the left auricle of the animal, an opening is made in the tympanic bulla using a diamond drill to give access to the inner ear. The labyrinth cavity is approached by an opening created above the oval window. This precisely-made opening exposes cranial nerve pair VII which are sectioned at the postganglionic level. The internal auditory meatus is obturated with a cicatrizing gelatin sponge and the surface tissues are restitched. The animals are given analgesics for 48 hours and antibiotics for five days postoperative.

After the vestibular nerve is sectioned, the success of the lesion can be evaluated by the severe deviation of the eyes (from the lesioned side downward, for the ipsilateral eye; from the unlesioned side upward, for the contralateral eye). Once the animal awakes, observations include strong spontaneous vestibular nystagmus whose rapid phase beats on the unlesioned side, postural asymmetry of the fore and hind limbs which are in hypertonic extension on the lesioned side, and heat tilt toward the lesioned side, occasionally combined with head nystagmus. The animal lies on the lesioned side, unable to assume an upright position. When the animal uprights itself, its support polygon, considerably enlarged, irremediably leads to the animal falling on the lesioned side. When the animal gains some ability to move about its environment, its progress deviates toward the lesioned side and it falls often.

### 1.1.2. Animal Treatments

The animals are divided into four groups comprising three treatment groups and one untreated control group, as follows:
control group (five cats), untreated after vestibular lesion but receiving a placebo,
experimental group one (four cats), treated with the racemic compound (acetyl-DL-leucine),
experimental group two (four cats), treated with the first enantiomer (acetyl-L-leucine),
experimental group three (four cats), treated with the second enantiomer (acetyl-D-leucine).

Pharmacological treatments for experimental groups one, two and three begin on the day of the lesion and continue until complete recovery (45 days for untreated control animals). In these three lesioned groups, treatment is administered by intravenous (IV) route during the first three days post-lesion and is followed by oral route (OR) treatment until recovery is complete. The doses administered are 30 mg/kg/day IV then 60 mg/kg/day OR for the racemate, and 15 mg/kg/day IV then 30 mg/kg/day OR for each of the two enantiomers. For the oral route, the substance is mixed with food; for the IV route, injection takes place after local anesthesia.

This protocol has the advantage of imitating the dosing schedule used in man in the acute and chronic treatment of vertigo, taking into account the absolute bioavailability of 45% observed for oral forms compared to IV forms.

For the control group, the placebo is also administered by intravenous route during the first three days post-lesion.

### 1.1.3. Behavioral Analysis Methods

a) Measurement of the Support Polygon

Support polygon surface area is a good indicator of the degree of postural stability in the cat. In general, it is quite small in the normal animal (roughly 50 cm²). It increases considerably, by four to eight times, after a unilateral vestibular lesion. This increase in polygon surface area reflects tonic asymmetries in the extensor and flexor muscles of the fore and hind feet and the loss of certain static equilibrium reflexes (Magnus reflexes, for example).

Thus, postoperative evolution of this indicator is a good measure of the animal's static equilibrium capacity. In addition, this indicator has prognostic value with respect to dynamic equilibrium performance, as measured by the rotating beam test.

Support polygon surface area measurements are taken with the animal in an upright position on all four legs, at rest, using an automated three-dimensional movement analysis system with virtual markers (Codamotion optoelectronic system coupled with a SIMI alignment device). Surface area measurements (in cm²) taken during the post-lesion period are standardized with respect to pre-lesion values. Thus, each animal acts as its own control (unit equivalent). This method enables direct between-group comparisons and within-group averaging.

b) Post-Lesion Horizontal Nystagmus Measurements

Recovery of oculomotor functioning is quantified by measuring post-operative regression of spontaneous vestibular nystagmus to light. This nystagmus is recorded in the horizontal plane by a video camera system that records eye movements (SIMI system). Nystagmus frequency is determined by the number of beats per unit time (10 seconds). Recordings are made daily until spontaneous nystagmus disappears. Experimental sessions do not exceed 15 minutes each and take place at the same time of day in order to control for possible variations attributable to the animal's vigilance.

c) Kinetic Equilibrium Functioning

The rotating beam test, as described by Xerri and Lacour (Xerri C, Lacour M: Compensation for postural and kinetic deficits following unilateral vestibular neurectomy in the cat. Role of sensory-motor activity [Compensation des déficits posturaux et cinétiqtues après neurectomie vestibulaire unilatérale chez le chat. Rôle de l'activité sensori-motrice], Acta Otolaryngol (Stockh) (1980) [in French], 90, 414-424) makes it possible to quantify kinetic equilibrium functioning deficits and recovery as a function of postoperative time.

Two compartments are connected by a cylindrical beam 3 m in length and 12 cm in diameter, placed 1.2 m above the floor. The beam can turn around its central axis with linear tangential velocities varying from 0 m/min to 37 m/min. Before the unilateral vestibular lesion (preoperative period), the cats are conditioned to move along this beam. Their maximum performance (MP), which corresponds to the highest beam rotation velocity not causing the animal to fall, is determined for four consecutive tests. In general, eight to 12 daily training sessions of approximately one hour are adequate for the animal to reach its MP. Inter-animal MP variations are relatively small (extreme values recorded: 27 m/min to 37 m/min; mean: 33 m/min; standard deviation: 2.08 m/min). For each cat, MP values obtained following unilateral vestibular neurectomy are expressed as a percentage of MP recorded at the end of training during the preoperative period.

Statistical analyses of the results are carried out using analysis of variance (Super Anova).

### 1.1.4. Results

a) Support Polygon

Results are presented in FIG. 1.

Animals treated with acetyl-D-leucine have an increased support polygon surface area identical to that observed in the control animals two days post-lesion; evolution of the surface area until its return to normal 40 days post-lesion is also identical to that observed in the control animals. Thus, acetyl-D-leucine does not have any beneficial effect on this parameter.

On the other hand, animals treated with acetyl-L-leucine have a significantly smaller support polygon surface area compared to that of the control animals and the support polygon surface area returns to normal 16 days post-lesion.

Acetyl-L-leucine used in a ½ dose has activity greater than or equal to that of acetyl-DL-leucine and it accelerates and supports compensation for postural deficits in lesioned animals.

b) Post-Lesion Horizontal Nystagmus

Results are presented in FIG. 2.

Animals treated with acetyl-D-leucine exhibit nystagmus whose frequency is identical to that of nystagmus observed in the control animals, with nystagmus disappearing eight days post-lesion. Thus, acetyl-D-leucine does not have any beneficial effect on this parameter.

On the other hand, animals treated with acetyl-L-leucine have nystagmus whose frequency is lower compared to that of nystagmus observed in the control animals, with nystagmus disappearing four days post-lesion.

Acetyl-L-leucine used in a ½ dose has activity greater than or equal to that of acetyl-DL-leucine and it accelerates and supports compensation for ocular nystagmus in lesioned animals.

c) Kinetic Equilibrium Functioning

Results are presented in FIG. 3.

Compensation for kinetic equilibrium in animals treated with acetyl-D-leucine is identical to that observed in the control animals, with a return to maximum performance (MP) 42 days post-lesion. Thus, acetyl-D-leucine does not have any beneficial effect on this parameter.

On the other hand, compensation for kinetic equilibrium in animals treated with acetyl-L-leucine is much more rapid than in the control animals, with a return to maximum performance (MP) 18 days post-lesion.

Acetyl-L-leucine used in a ½ dose has activity greater than or equal to that of acetyl-DL-leucine and it accelerates and supports compensation for kinetic equilibrium in lesioned animals.

### EXAMPLE 2

### Compared Effects of a Pharmaceutical Treatment with Acetyl-DL-Leucine and with its L Isomer in the Compensation of Vestibular Deficits

### 2.1. Protocol

### 2.1.1. Vestibular Neurectomy

The experiment involves 18 cats from the breeder IFA-CREDO (France). The cats undergo a unilateral vestibular neurectomy of the left side, as in example 1.

### 2.1.2. Animal Treatments

The animals are divided into three groups comprising one group treated with racemic coumpound (acetyl-DL-leucine) (groups 1) and two treated with acetyl-L-leucine (groups 2 and 3), as follows:
experimental group one (six cats), treated after vestibular lesion with the racemic compound (acetyl-DL-leucine) at 30 mg/kg per day,
experimental group two (six cats), treated after vestibular lesion with the L enantiomer (acetyl-L-leucine) at 15 mg/kg per day,
experimental group three (six cats), treated after vestibular lesion with the L enantiomer (acetyl-L-leucine) at 7.5 mg/kg per day,

Pharmacological treatments for experimental groups 1 to 3 begin on the day of the lesion. Treatment is administered by intravenous (IV) route during the first three days post-lesion.

### 2.1.3. Results

Support Polygon

Results are presented in FIG. 4.

Surprisingly, acetyl-L-leucine proved to efficiently restore the postural, locomotor and oculomotor functions deteriorated by a vestibular lesion.

1.-7. (canceled)

8. A method of treating vestibular neuritis which comprises administering to a patient in need thereof an effective amount of acetyl-L-leucine and the pharmaceutically acceptable salts.

9. The method according to claim 8, wherein the acetyl-L-leucine is a mixture chosen among mixtures comprising at least 95%-100% acetyl-L-leucine, 96%-100% acetyl-L-leucine, 97%-100% acetyl-L-leucine, 98% 100% acetyl-L-leucine, 99%-100% acetyl-L-leucine or 100% acetyl-L-leucine.

10. The method according to claim 8 or 9, wherein the acetyl-L-leucine is administered by oral route or intravenous route.

11. The method according to claim 8, wherein the acetyl-L-leucine is administered by oral route in a dose between 100 mg and 20 g per day.

12. The use method according to claim 8, wherein the acetyl-L-leucine is administered by intravenous route in a dose between 100 mg and 2 g per day without interruption.

13. The method according to claim 11, wherein the acetyl-L-leucine is administered by oral route in a dose between 100 mg and 4 g per day.
